(19) United States
(12) Patent Application Publication
(10) Pub. No.: US 2025/0090240 A1
(43) Pub. Date: Mar. 20, 2025

(54) NAVIGATION ADAPTER CUFF FOR ENT INSTRUMENT

(71) Applicant: Acclarent, Inc., Irvine, CA (US)

(72) Inventors: Gigi KOE, Anaheim, CA (US); Alex GONZALEZ, Lake Forest, CA (US); Tatyana TEICHNER, Haifa (IL); Tamir YELLIN, Haifa (IL); Raymond Yue-sing TANG, Rosemead, CA (US); George GUSEIN, Acre (IL); Erez MARDO, Mikhmoret (IL)

(21) Appl. No.: 18/885,721

(22) Filed: Sep. 15, 2024

Related U.S. Application Data
(60) Provisional application No. 63/538,527, filed on Sep. 15, 2023.

(51) Int. Cl. A61B 34/20; A61B 17/00; A61B 17/32

(52) U.S. Cl. CPC A61B 34/20; A61B 17/32002; A61B 2017/00486; A61B 2034/2051

(57) ABSTRACT

Navigation sensors can indicate a real-time location of a surgical instrument within a patient. An adapter or other component that can be readily coupled with a surgical instrument and has one or more integral navigation sensors. The adapter can include an adapter body, an instrument coupling arm, and one or more navigation sensors. The adapter body extends between a proximal end and a distal end of the adapter. The instrument coupling arm is configured to non-removably engage a portion of the instrument. The one or more navigation sensors are configured generate signals indicating a position of at least a portion of the instrument in three-dimensional space and can be arranged in various configurations about the adapter body.

### CROSS-REFERENCE TO RELATED APPLICATIONS

This application claims the benefit of U.S. Provisional Application No. 63/538,527, filed Sep. 15, 2023, the entirety of which is incorporated herein by reference.

### BACKGROUND

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. An example of an electromagnetic IGS navigation system that may be used in IGS procedures is the TRUDI® Navigation System by Acclarent, Inc., of Irvine, California. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, some instruments can include sensors (e.g., electromagnetic coils that emit electromagnetic fields and/or are responsive to externally generated electromagnetic fields), which can be used to perform the procedure while the sensors send data to the computer indicating the current position of each sensor-equipped instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

In some instances, it may be desirable to guide an instrument using IGS techniques described above. However, certain IGS navigation systems may be equipped for use with specific instruments and certain instruments may be equipped for use with specific IGS navigation systems or no IGS navigation system at all. Thus, it may be desirable to provide an easily implemented and cost-effective means to incorporate compatibility with a given IGS navigation system into a variety of instruments that otherwise lack position sensing features. While several systems and methods have been made and used in connection with IGS navigation stems, it is believed that no one prior to the inventors has made or used the inventions described in the appended claims.

### BRIEF DESCRIPTION OF THE DRAWINGS

The drawings and detailed description that follow are intended to be merely illustrative and are not intended to limit the scope of the inventions as contemplated by the inventors.

FIG. 1 depicts a schematic view of an example of a IGS navigation system being used on a patient seated in an example of a medical procedure chair, in accordance with some embodiments.

FIG. 2 depicts a perspective view of an example of a tissue shaving instrument and an example of an adapter cuff for use in combination with the IGS navigation system of FIG. 1 and the instrument, in accordance with some embodiments.

FIG. 3 depicts a perspective view of the adapter cuff of FIG. 2, in accordance with some embodiments.

FIG. 4 depicts a sectional view of the adapter cuff of FIG. 2, taken along line 4-4 of FIG. 2, in accordance with some embodiments.

FIG. 5 depicts a distal end view of the adapter cuff of FIG. 2, in accordance with some embodiments.

FIG. 6A depicts a cross-sectional view of the adapter cuff of FIG. 2 being initially coupled to the instrument of FIG. 2, with the adapter cuff in a distal position, in accordance with some embodiments.

FIG. 6B depicts a cross-sectional view of the adapter cuff of FIG. 2 being coupled to the instrument of FIG. 2, with the adapter cuff in an intermediatory position, in accordance with some embodiments.

FIG. 6C depicts a cross-sectional view of the adapter cuff of FIG. 2 coupled to the instrument of FIG. 2, in accordance with some embodiments.

FIG. 7 depicts a top perspective view of the adapter cuff of FIG. 2 coupled to the instrument of FIG. 2, in accordance with some embodiments.

FIG. 8 depicts an exploded perspective view of another example of an adapter cuff for use in combination with the IGS navigation system of FIG. 1 and the instrument of FIG. 2, in accordance with some embodiments.

FIG. 9 depicts a bottom perspective view of the adapter cuff of FIG. 8, in accordance with some embodiments.

FIG. 10 depicts a sectional view of the adapter cuff of FIG. 8, in accordance with some embodiments.

FIG. 11A depicts a cross-sectional view of the adapter cuff of FIG. 8 being initially coupled to the instrument of FIG. 2, with the adapter cuff in a distal position, in accordance with some embodiments.

FIG. 11B depicts a cross-sectional view of the adapter cuff of FIG. 8 being coupled to the instrument of FIG. 2, with the adapter cuff in an intermediatory position, in accordance with some embodiments.

FIG. 11C depicts a cross-sectional view of the adapter cuff of FIG. 8 coupled to the instrument of FIG. 2, in accordance with some embodiments.

FIG. 12 depicts a top perspective view of the adapter cuff of FIG. 8 coupled to the instrument of FIG. 2, in accordance with some embodiments.

FIG. 13 depicts a perspective view of another example of an adapter cuff for use in combination with the IGS navigation system of FIG. 1 and the instrument of FIG. 2, in accordance with some embodiments.

FIG. 14 depicts an exploded perspective view of another example of an adapter cuff for use in combination with the IGS navigation system of FIG. 1 and the instrument of FIG. 2, in accordance with some embodiments.

FIG. 15 depicts another exploded perspective view of the adapter cuff of FIG. 14, in accordance with some embodiments.

FIG. 16 depicts an elevational side view of another example of an adapter cuff for use in combination with the IGS navigation system of FIG. 1 and a surgical drilling instrument, in accordance with some embodiments.

FIG. 17 depicts a front plan view of the adapter cuff of FIG. 16, in accordance with some embodiments.

FIG. 18 depicts an elevational side view of the adapter cuff of FIG. 16 operatively attached to a surgical drilling instrument, in accordance with some embodiments.

FIG. 19 depicts an elevational side view of another example of an adapter cuff for use in combination with the IGS navigation system of FIG. 1 and a surgical drilling instrument of FIG. 18, in accordance with some embodiments.

FIG. 20 depicts a front plan view of the adapter cuff of FIG. 19, in accordance with some embodiments.

FIG. 21 depicts an elevational side view of the adapter cuff of FIG. 16 operatively attached to the surgical drilling instrument of FIG. 18, in accordance with some embodiments.

FIG. 22 depicts an elevational side view of another example of an adapter cuff for use in combination with the IGS navigation system of FIG. 1 and a surgical drilling instrument of FIG. 18, in accordance with some embodiments.

FIG. 23 depicts a front plan view of the adapter cuff of FIG. 22, in accordance with some embodiments.

FIG. 24 depicts an elevational side view of the adapter cuff of FIG. 22 operatively attached to the surgical drilling instrument of FIG. 18, in accordance with some embodiments.

FIG. 25 depicts an elevational side view of another example of an adapter cuff for use in combination with the IGS navigation system of FIG. 1 and a surgical drilling instrument of FIG. 18, in accordance with some embodiments.

FIG. 26 depicts a front plan view of the adapter cuff of FIG. 25, in accordance with some embodiments.

FIG. 27 depicts an elevational side view of the adapter cuff of FIG. 25 operatively attached to the surgical drilling instrument of FIG. 18, in accordance with some embodiments.

### DETAILED DESCRIPTION

The following description of certain examples of the inventions should not be used to limit the scope of the present inventions. Other examples, features, aspects, embodiments, and advantages of the inventions will become apparent to those skilled in the art from the following description, which is by way of illustration, one of the best modes contemplated for carrying out the inventions. As will be realized, the inventions are capable of other different and obvious aspects, all without departing from the inventions. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. Example of an IGS Navigation System

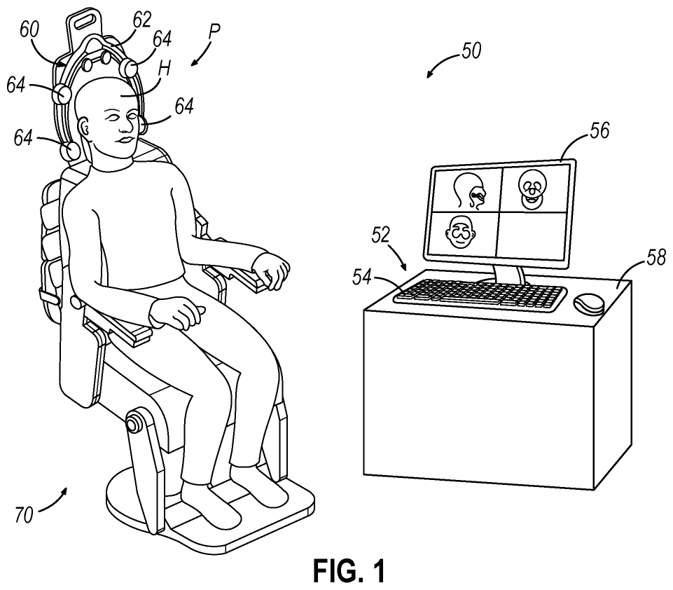

When performing a medical procedure within a head of a patient (P), it may be desirable to have information regarding the position of an instrument within the head (H) of the patient (P), particularly when the instrument is in a location where it is difficult or impossible to obtain an endoscopic view of a working element of the instrument within the head of the patient (P). FIG. 1 shows an example of an the IGS navigation system 50 enabling a medical procedure to be performed within a head (H) of a patient (P) using image guidance. In addition to or in lieu of having the components and operability described herein the IGS navigation system 50 may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010; and/or U.S. Pat. No. 11,065,061, entitled “Systems and Methods for Performing Image Guided Procedures within the Ear, Nose, Throat and Paranasal Sinuses,” issued Jul. 20, 2021. The disclosures of U.S. Pat. No. 7,720,521, entitled “Methods and Devices for Performing Procedures within the Ear, Nose, Throat and Paranasal Sinuses,” issued May 18, 2010, and U.S. Pat. No. 11,065,061, entitled “Systems and Methods for Performing Image Guided Procedures within the Ear, Nose, Throat and Paranasal Sinuses,” issued Jul. 20, 2021, are incorporated by reference herein, in their entirety.

The IGS navigation system 50 of the present example comprises a field generator assembly 60, which comprises a set of magnetic field generators 64 that are integrated into a horseshoe-shaped frame 62. The field generators 64 are operable to generate alternating magnetic fields of different frequencies around the head (H) of the patient (P). An instrument may be inserted into the head (H) of the patient (P). Such an instrument may include one or more position sensors as described in greater detail below. In the present example, the frame 62 is mounted to a chair 70, with the patient (P) being seated in the chair 70 such that the frame 62 is located adjacent to the head (H) of the patient (P). By way of example only, the chair 70 and/or the field generator assembly 60 may be configured and operable in accordance with at least some of the teachings of U.S. Pat. No. 10,561,370, entitled “Apparatus to Secure Field Generating Device to Chair,” Issued Feb. 18, 2020. The disclosure of U.S. Pat. No. 10,561,370, entitled “Apparatus to Secure Field Generating Device to Chair,” Issued Feb. 18, 2020, is incorporated by reference herein, in its entirety. In some other variations, the patient (P) lies on a table; and the field generator assembly 60 is positioned on or near the table.

The IGS navigation system 50 of the present example further comprises a processor 52, which controls the field generators 64 and other elements of the IGS navigation system 50. For instance, the processor 52 is operable to drive the field generators 64 to generate alternating electromagnetic fields; and process signals from the instrument to determine the location of a position sensor in the instrument within the head (H) of the patient (P). The processor 52 comprises a processing unit (e.g., a set of electronic circuits arranged to evaluate and execute software instructions using combinational logic circuitry or other similar circuitry) communicating with one or more memories. The processor 52 of the present example is mounted in a console 58, which comprises operating controls 54 that include a keypad and/or a pointing device such as a mouse or trackball. A physician uses the operating controls 54 to interact with the processor 52 while performing the surgical procedure.

While not shown, the instrument may include a position sensor that is responsive to positioning within the alternating magnetic fields generated by the field generators 64. A coupling unit (not shown) may be secured to the proximal end of the instrument and may be configured to provide communication of data and other signals between the console 58 and the instrument. The coupling unit may provide wired or wireless communication of data and other signals.

In some versions, the position sensor of the instrument may comprise at least one coil at or near the distal end of the instrument. When such a coil is positioned within an alternating electromagnetic field generated by the field generators 64, the alternating magnetic field may generate electrical current in the coil, and this electrical current may be communicated along the electrical conduit(s) in the instrument and further to the processor 52 via the coupling unit. This phenomenon may enable the IGS navigation system 50 to determine the location of the distal end of the instrument within a three-dimensional space (i.e., within the head (H) of the patient (P), etc.). To accomplish this, the processor 52 executes an algorithm to calculate location coordinates of the distal end of the instrument from the position related signals of the coil(s) in the instrument. Thus, a navigation sensor may serve as a position sensor by generating signals indicating the real-time position of the sensor within three-dimensional space.

The processor 52 uses software stored in a memory of the processor 52 to calibrate and operate the IGS navigation system 50. Such operation includes driving the field generators 64, processing data from the instrument, processing data from the operating controls 54, and driving the display screen 56. In some implementations, operation may also include monitoring and enforcement of one or more safety features or functions of the IGS navigation system 50. The processor 52 is further operable to provide video in real time via the display screen 56, showing the position of the distal end of the instrument in relation to a video camera image of the patient's head (H), a CT scan image of the patient's head (H), and/or a computer-generated three-dimensional model of the anatomy within and adjacent to the patient's nasal cavity. The display screen 56 may display such images simultaneously and/or superimposed on each other during the surgical procedure. Such displayed images may also include graphical representations of instruments that are inserted in the patient's head (H), such that the operator may view the virtual rendering of the instrument at its actual location in real time. By way of example only, the display screen 56 may provide images in accordance with at least some of the teachings of U.S. Pat. No. 10,463,242, entitled “Guidewire Navigation for Sinuplasty,” issued Nov. 5, 2019. The disclosure of U.S. Pat. No. 10,463,242, entitled “Guidewire Navigation for Sinuplasty,” issued Nov. 5, 2019, is incorporated by reference herein, in its entirety. In the event that the operator is also using an endoscope, the endoscopic image may also be provided on the display screen 56.

The images provided through the display screen 56 may help guide the operator in maneuvering and otherwise manipulating instruments within the patient's head (H). It should also be understood that other components of a surgical instrument and other kinds of surgical instruments, as described below, may incorporate a navigation sensor like the navigation sensor described above.

### II. Example of a Tissue Shaving Instrument

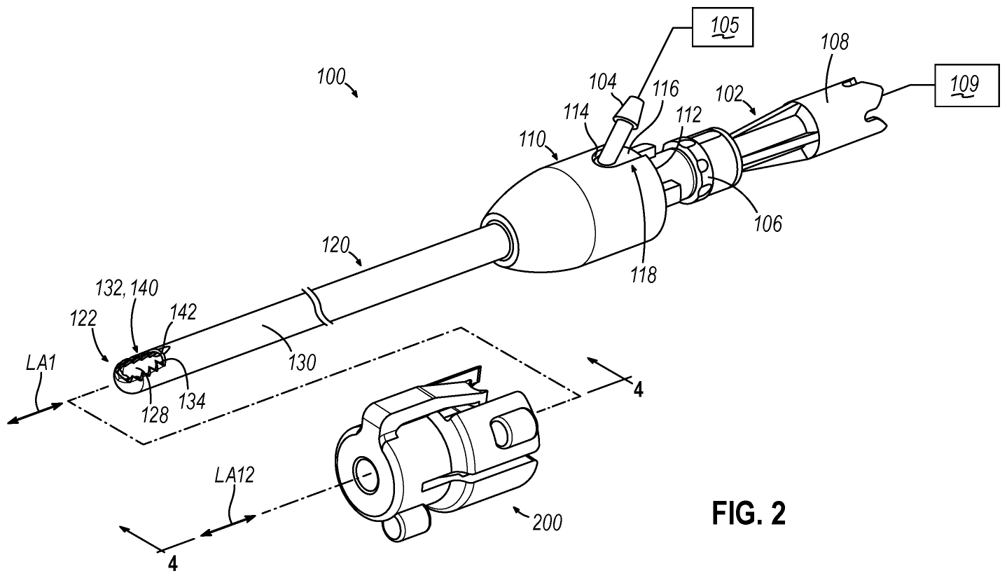

FIG. 2 shows an example of a tissue shaving instrument 100, which is configured for use in a surgical procedure (e.g., a FESS procedure, a turbinate reduction procedure, etc.) where bone and/or soft tissue is removed from within the car, nose, or throat of a patient. As will be described in greater detail below, it may be desirable to adapt the tissue shaving instrument 100 or other instruments for use with the IGS navigation system 50 because the tissue shaving instrument 100 itself may lack position sensors configured for use with the IGS navigation system 50. While the example described below is in the context of the tissue shaving instrument 100, the teachings below may be readily applied in the contexts of other instruments, including but not limited to bur instruments, drill instruments, suction instruments, and other instruments as will be apparent to those skilled in the art in view of the teachings herein.

The tissue shaving instrument 100 of this example includes a proximal hub assembly 102, a distal hub 110, and a shaft assembly 120. The distal hub 110 is configured to be fixedly secured to a body (not shown); while the proximal hub assembly 102 is configured to be inserted into the body. By way of example only, the body may comprise a handle that is configured for grasping by an operator. The distal hub 110 includes a fluid port 104 that is configured to couple with a fluid source 105. In some examples, the fluid port 104 and the fluid source 105 are configured to apply suction to draw tissue and fluids during operation of the tissue shaving instrument 100. In addition, or in the alternative, the fluid port 104 and the fluid source 105 may be configured to communicate fluids to irrigate patient anatomy during operation of the tissue shaving instrument 100.

The proximal hub assembly 102 is rotatable relative to the distal hub 110. In the present example, the proximal hub assembly 102 is coupled with a multi-function control unit 109. The multi-function control unit 109 is configured to communicate power, signals, and/or fluid for the operation of the tissue shaving instrument 100. For instance, in some examples, the multi-function control unit 109 is configured to provide power to a motor (not shown) that is disposed in in the same body to which the distal hub 110 is coupled; and in which the proximal hub assembly 102 is disposed. Such a motor may be operable to drive one or more components of the proximal hub assembly 102, to thereby drive rotation of a cutting shaft 128 of the shaft assembly 120 as will be described in greater detail below. As another variation, the motor may be positioned remotely from the body to which the distal hub 110 is coupled, and in which the proximal hub assembly 102 is disposed, and a rotary drive cable may be coupled with the proximal hub assembly 102 to communicate rotary motion from the remote motor to the tissue shaving instrument 100. In some versions, the multi-function control unit 109 is also configured to provide suction to the tissue shaving instrument 100. Such suction may be used to draw tissue or fluids during operation of the tissue shaving instrument 100. In some versions, the multi-function control unit 109 is configured to activate the fluid source 105.

The distal hub 110 is configured to couple with a body (e.g., handle) as described above; and in some versions this coupling is removable such that the distal hub 110 may be decoupled from the body after being coupled with the body. The hub 110 defines a generally cylindrical shape with the fluid port 104 extending from a portion thereof. As noted herein, the fluid port 104 may be in communication with a portion of the shaft assembly 120 via the distal hub 110.

The distal hub 110 further includes a boundary surface 114 extending distally from a proximal end 112 of the distal hub 110. In the current example, the boundary surface 114 includes two linear portions extending distally from the proximal end 112, where the two linear portions are connected together by a distal arched surface. The boundary surface 114 extends inwardly into an offset floor 116. Together, the boundary surface 114 and the offset floor 116 define a recessed pocket 118. The fluid hub 104 extends upwardly from the offset floor 116 such that a portion of the fluid hub 104 is contained within the confines of the boundary surface 114, and thereby within the recessed pocket 118. As will be described in greater detail below, the fluid hub 104, the boundary surface 114, and/or the offset floor 116 may be used as location features to index accessory components relative to the distal hub 110 and/or other features of the tissue shaving instrument 100. It should be understood that the recessed pocket 118, the boundary surface 114, and the offset floor 116 may have any suitable geometry as would be apparent to one skilled in the art in view of the teachings herein.

The shaft assembly 120 extends distally from the distal hub 110 and the proximal hub assembly 102. The shaft assembly 120 includes an inner shaft 128 and an outer shaft 130. In the present example, both the inner shaft 128 and the outer shaft 130 are coaxial with each other and define a substantially similar shape that is entirely straight (i.e., extending along a longitudinal axis). In some other variations, both the inner shaft 128 and the outer shaft 130 may include a common bent portion.

The outer shaft 130 generally extends from hub 110, while the inner shaft 128 extends from proximal hub assembly 102. In this configuration, fluids may be communicated via hub 110 and fluid port 104 through the interior of the outer shaft 130, along a radially extending gap defined between the inner shaft 128 and the outer shaft 130. Such fluids may be communicated to apply irrigation or suction during use of the tissue shaving instrument 100. Additionally, both the hub 110 and the outer shaft 130 may be removably secured to the proximal hub assembly 102 and or the inner shaft 128 such that the hub 110 and the outer shaft 130 may be removable.

An end effector 122 is formed at the distal end of the shaft assembly 120. The end effector 122 includes a first transverse opening 132 formed at the distal end of the outer shaft 130 and a second transverse opening 140 formed at the distal end of the inner shaft 128. Transverse openings 132, 140 share a common longitudinal position along the shaft assembly 120 with each other. The transverse openings 132, 140 may alternate between being angularly aligned with each other and being angularly offset from each other as the inner shaft 128 is rotated relative to the outer shaft 130 by the proximal hub assembly 102. The first transverse opening 132 is bounded by a first edge 134 while the second transverse opening 140 is bounded by a second edge 142. Edges 134, 142 cooperate to shear tissue that protrudes into the openings 132, 140 as the inner shaft 128 is rotated relative to the outer shaft 130. One or both of edges 134, 142 may include serrations or may be otherwise sharp to promote cutting of tissue by the end effector 122. During such rotation, suction is applied via a lumen defined by the inner shaft 128 to draw tissue into the transverse opening 140, the edges 134, 142 shear the tissue, and the sheared tissue is drawn proximally through the lumen of the inner shaft 128 under the influence of the suction.

### III. Example of a Navigation Adapter for Providing Navigation to an Instrument

Those skilled in the art will understand that it may be beneficial to have data from navigation sensors indicating the real-time location of the end effector 122 within the patient (P) during operation of the tissue shaving instrument 100. This may be particularly desirable in scenarios where the end effector 122 is used within the head of the patient, where endoscopic visualization may be difficult, and where sensitive anatomical features present very little margin for error in positioning of the end effector 122. To that end, it may be desirable to provide an adapter or other component having one or more integral navigation sensors. Such an adapter may be readily coupled with an instrument, such as the tissue shaving instrument 100 described above, that otherwise lacks any navigation sensors. In such a scenario, the adapter may impart navigation capabilities to the instrument.

Similarly, it may be desirable to use an adapter having one or more integral navigation sensors in combination with an instrument that already has one or more navigation sensors, where the position data from the one or more navigation sensors of the adapter may supplement the position data from the one or more navigation sensors of the instrument. In such a scenario, the adapter may enhance navigation capabilities of the instrument. In either of the above scenarios, the adapter may be configured to avoid adding bulk to the instrument; and to be easily assembled with the instrument in the surgical field or operating room. Moreover, the adapter may be configured to accommodate different kinds of instruments and/or instruments otherwise having different outer diameters, such that the adapter need not necessarily be limited in its compatibility to just one single instrument size or type.

FIGS. 2-5 show an example of an adapter, which may provide the above-described benefits and functionality, in the form of an adapter cuff 200. In the present example, the adapter cuff 200 is configured to couple with the tissue shaving instrument 100. As will be described in greater detail below, adapters similar to the adapter cuff 200 of the present example may be used in combination with instruments similar to the tissue shaving instrument 100 to adapt such instruments for navigation using navigation systems such as the IGS navigation system 50. In particular, existing instruments may lack features such as one or more integral navigation sensors to facilitate navigation of such instruments via systems such as the IGS navigation system 50. Therefore, it may be desirable to add navigation capabilities to such instruments via components such as adapters. Such a combination may be desirable to facilitate the use of certain virtual reality features and/or advanced mapping features. Additionally, such a combination may be desirable to facilitate real-time overlay of positional data on images, providing true anatomical measurements that may improve workflows and/or procedure outcomes. Although certain combinations of adapters and instruments are described herein, it should be understood that such combinations may be combined with other adapters and/or instruments to promote use of the IGS navigation system 50 with multiple instruments used in a given procedure.

The adapter cuff 200 of the present example includes a cuff body 210, a coupling arm 220, a position sensor assembly 230, a cable 202, and a connector 204. As will be described in greater detail below, the adapter cuff 200 is generally configured to mate with the tissue shaving instrument 100 in such a way to consistently orient the adapter cuff 200 relative to the tissue shaving instrument 100; as well as prevent decoupling of the adapter cuff 200 from the tissue shaving instrument 100. Once suitably coupled, the adapter cuff 200 may add, or otherwise supplement, navigation capabilities with respect to the tissue shaving instrument 100. Thus, the position sensor assembly 230 of the adapter cuff 200 is configured for use in combination with the IGS navigation system 50.

As will also be described in greater detail below, position sensors 234 of the position sensor assembly 230 are configured to locate one or more geometric features of the adapter cuff 200 in three-dimensional space using the IGS navigation system 50. With the adapter cuff 200 suitably attached to the shaving the instrument 100 in accordance with the description herein, such geometric features of the adapter cuff 200 can then be correlated with one or more features of the tissue shaving instrument 100 (e.g., the end effector 122, and more particularly the first transverse opening 132, etc.) to likewise locate one or more features of the tissue shaving instrument 100 in three-dimensional space.

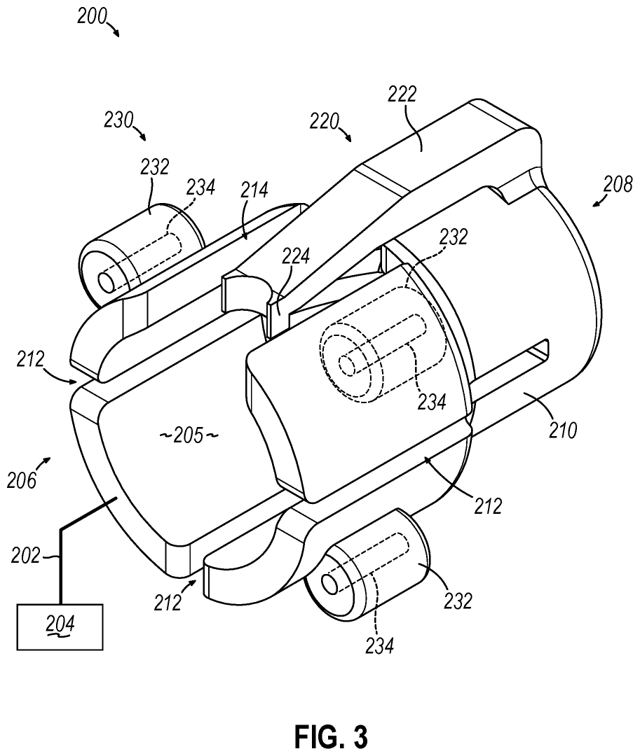

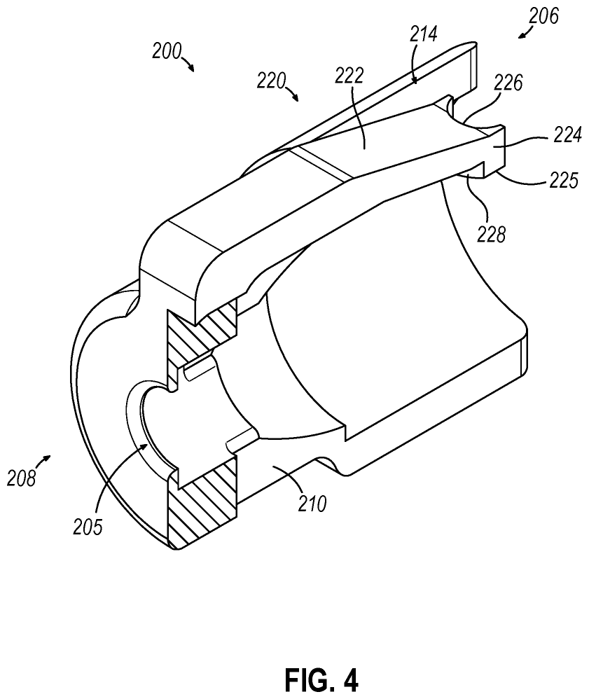

Turning to FIGS. 3-4, the cuff body 210 extends between a proximal end 206 and a distal end 208. The cuff body 210 also defines a through hole 205 extending between the proximal end 206 and the distal end 208. The through hole 205 is dimensioned to receive the shaft assembly 120 in order to couple the adapter cuff 200 with the instrument 100 such that the distal end 208 of the cuff body 210 is adjacent to a proximal end of the shaft assembly 120, while the proximal end 206 of the cuff body 210 suitably houses at least a portion of the distal hub 110 of the instrument 100. Therefore, as indicated in FIG. 2, in order to attach the cuff body 210 to the distal hub 110 in accordance with the description herein, an operator may insert the end effector 122 into the through hole 205 via the proximal end 206 of the cuff body 210 and actuate the cuff body 210 proximally relative to the shaft assembly 120.

The cuff body 210 also defines a plurality of the longitudinally extending slots 212. In some instances, the longitudinally extending slots 212 are dimensioned to receive longitudinally extending ribs and/or protrusions that are disposed on an outer surface of the distal hub 110 of the instrument 100. In some instances, the longitudinally extending slots 212 may further promote radial flexibility of the cuff body 210 such that as the cuff body 210 initially receives the distal hub 110 of the instrument 100, the cuff body 210 may flare radially outward to accommodate receiving the distal hub 110.

The cuff body 210 also defines an arm housing window 214, which the hub coupling arm 220 is disposed within. As will be described in greater detail below, the hub coupling arm 220 is configured to engage the distal hub 110 of the instrument 100 in order to consistently orient the adapter cuff 200 relative to the tissue shaving instrument 100. Providing consistent orientation of the adapter cuff 200 relative to the distal hub 100 may allow the position sensor assembly 230 to locate one or more features of the tissue shaving instrument 100 (e.g., the end effector 122, and more particularly the first transverse opening 132, etc.) in three-dimensional space using the IGS navigation system 50. As will also be described in greater detail below, the hub coupling arm 220 is configured to prevent the adapter cuff 200 from decoupling with the distal hub 110 after the adapter cuff 200 has been fully seated on the distal hub 110.

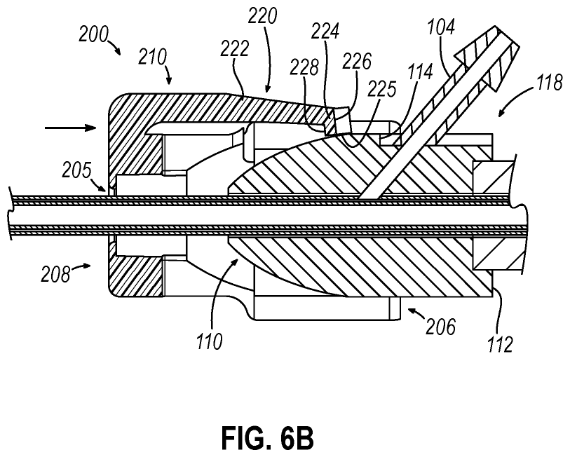

The hub coupling arm 220 includes a resilient arm 222 that extends proximally from the distal end 208 into the arm housing window 214. The resilient arm 222 terminates proximally into a distal hub engagement body 224. The distal hub engagement body 224 includes a complementary profile dimensioned to fit within a suitable portion of the pocket recess 118 (see FIG. 2) when the adapter cuff 200 is operatively attached to the distal hub 110 (see FIG. 7). In the current example, the distal hub engagement body 224 includes an offset floor engagement surface 225, a port engagement surface 226, and a recess boundary engagement surface 228.

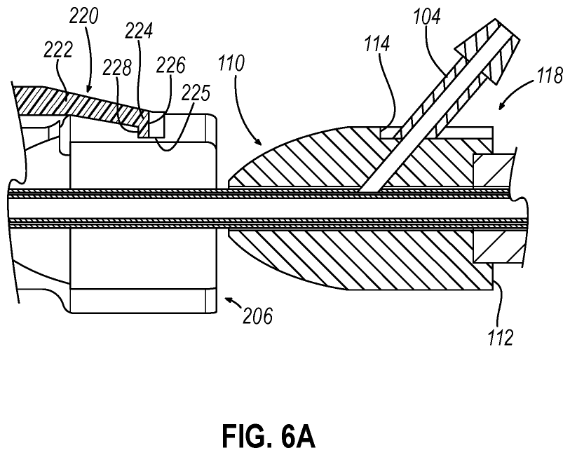

The resilient arm 222 is configured to deflect between a relaxed position (see FIGS. 3-4, 6A, and 6C) and a flexed position (see FIG. 6B). The resilient arm 222 is resiliently biased toward the relaxed position. FIGS. 6A-6C show an example of coupling of the adapter cuff 200 and the distal hub 110 of the instrument 100. First, as mentioned above, the proximal end 206 of the adapter cuff 200 may be slid over the end effector 122 and the shaft assembly 120 of the instrument 100 via the through hole 205 to the position shown in FIG. 6A. Next, the adapter cuff 200 may be further slid proximally as shown between FIGS. 6A-6B. When the adapter cuff 200 is inserted over the shaft assembly 120 onto the distal hub 110, the resilient arm 222 deflects into the flexed position in response to the distal hub engagement body 224 engaging the outer surface of the distal hub 110. As mentioned above, the distal hub engagement body 224 includes a complementary profile dimensioned to at least partially rest within the recessed pocket 118 defined by the distal hub 110.

Once the hub engagement body 224 is longitudinally and laterally aligned with the recessed pocket 118, the resilient arm 222 may return toward the relaxed position, thereby driving the distal hub engagement body 224 into the confines of the recessed pocket 118 as shown in FIG. 6C and 7. The resilient arm 222 may drive the distal hub engagement body 224 into the confines of the recessed pocket 118 with sufficient force in order to provide tactile and/or audible feedback, thereby indicating to an operator that the cuff adapter 200 is suitably engaged with the distal hub 110.

When the distal hub engagement body 224 is housed within the suitable portion of the pocket recess 118, as shown in FIG. 6C, at least a portion of the port engagement surface 226 is directly adjacent to the fluid port 104, while at least a portion of the recess boundary engagement surface 228 is directly adjacent to the boundary surface 114 defining the recessed pocket 118. The offset floor engagement surface 225 may rest on the offset floor 116 of the distal hub 110 when the adapter cuff 200 is suitably coupled to the distal hub 110.

The port engagement surface 226 may contact the fluid port 104 in order to inhibit over insertion of the adapter cuff 200 proximally relative to the distal hub 110 when suitably coupled. Similarly, the boundary engagement surface 228 may contact suitable portions of the boundary surface 114 defining the recessed pocket 118 in order to inhibit distal movement of the adapter cuff 200 relative to the distal hub 110 when suitably coupled. The port engagement surface 226 and/or the boundary engagement surface 228 may also engage suitable portions of the fluid port 104 and/or the boundary surface 114, respectively, in order to inhibit the adapter cuff 200 from rotating about the coaxial longitudinal axis LA1, LA2 of the instrument 100 and the adapter cuff 200. Therefore, once the adapter cuff 200 is suitably coupled to the distal hub 110, the cuff 200 may be constrained to the consistent and predetermined orientation relative to the distal hub 110 and be prevented from decoupling from the distal hub 110 via portions of the distal hub engagement body 224 housed within the recessed pocket 118.

Ensuring the adapter cuff 200 couples with hub 110 in only one predetermined angular orientation, while also preventing the adapter cuff 200 from moving relative to the hub 110 during use, ensures the sensors 234 consistently have a particular alignment with the end effector 122. Such a particular alignment may be desirable to enhance locating the end effector 122, and particularly the first transverse opening 132, in three-dimensional space via the sensors 234 while using the IGS navigation system 50 in accordance with the description herein.

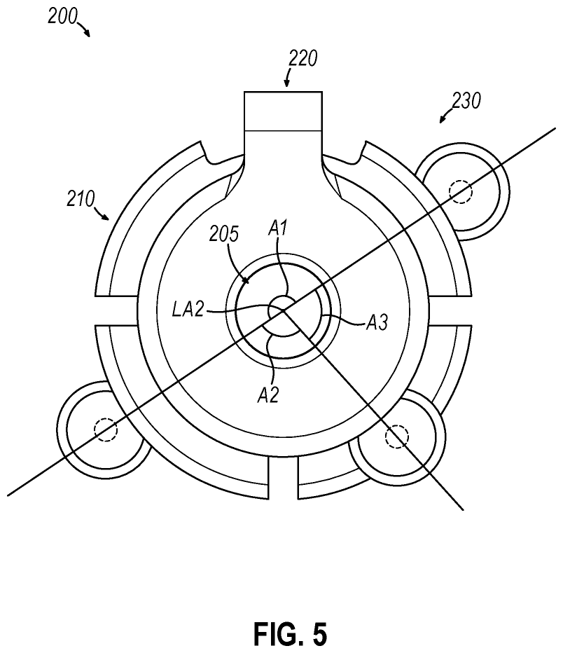

As shown in FIGS. 3 and 5, the navigation sensor assembly 230 includes three individual sensor housings 232, each housing a respective position sensor, such as a single axis sensor 234. The sensor housings 232 are fixedly attached to the cuff body 210 such that each single axis sensor 234 is also fixed relative to the cuff body 210. Each single axis sensor 234 may be secured to its respective sensor housing 232 using any suitable means as would be apparent to those skilled in the art in view of the teachings herein. For example, each single axis sensor 234 may be inserted into an opening defined by a respective sensor housing 232 and fixed within respective sensor housing 232 via adhesive, friction fitting, or both.

The single axis sensors 234 are in communication with the connector 204 via the cable 202. The connector 204 is configured to facilitate communication of the single axis sensors 234 with the console 58 of the IGS navigation system 50. Therefore, during an example of use, the single axis sensors 234 are in operative communication with the IGS navigation system 50 via the cable 202 and the connector 204. The connector 204 may establish communication with the console 58 via wired and/or wireless communication. Once the adapter cuff 200 is suitably attached to the instrument 100, the three single axis sensor 234 cooperatively function to locate one or more geometric features of the adapter cuff 200 in three-dimensional space using the IGS navigation system 50. With the adapter cuff 200 being consistently positioned relative to the distal hub 110 and the instrument 100 via the hub coupling arm 220, the IGS navigation system 50 may utilize the location of the adapter cuff 200 to correlate the position of one or more features of tissue saving instruments 100 (e.g., the end effector 122, and more particularly the first transverse opening 132, etc.) to likewise locate one or more features of the tissue shaving instrument 100 in three-dimensional space.

Therefore, the navigation sensors 234 may be used with the IGS navigation system 50 to navigate the tissue shaving instrument 100 through one or more anatomical passageways of a patient and/or relative to one or more anatomical features of a patient. As described above, such navigation may be combined with navigation of other instruments (e.g., probes) to facilitate real-time overlays of instrument positioning on visualizations. In some uses, such real-time overlays may be used to facilitate true anatomical measurements of patient anatomy, thereby improving procedure workflows and procedure outcomes.

The current example shows a single cable 202 in communication with the connector 204. Although not shown, it should be understood that communication between the sensors 234 and the cable 202 may be facilitated by a plurality of electrical communication paths extending through the sensor housing 232 and/or the cuff body 210. Such electrical communication paths may take on a variety of forms. For instance, in some examples, such electrical communication paths may be configured as traces on a flexible printed circuit board or flex circuit attached to or embedded within a portion of the cuff body 210. In other examples, such electrical communication paths may be configured as a plurality of wires extending through the cuff body 210. Such wires may likewise be attached to or embedded within a portion of the cuff body 210. Alternatively, in some examples, the cuff body 210 may be configured to define one or more dedicated lumens configured to receive one or more of such wires. Alternatively, each sensor 234 may have an individual cable 202 that each couple to the connector 204 individually; or meet at a juncture that couples to the connector 204. Of course, various other means of facilitating electrical communication paths will be apparent to those skilled in the art in view of the teachings herein.

It should be understood that when the adapter cuff 200 is suitably attached to the instrument 100 in accordance with the description herein, none of the three single axis sensors 234 are coaxial with the longitudinal axis LA1 (see FIG. 2) of the shaft assembly 120 of tissue shaving instruments 100. Therefore, it should be understood that the single axis sensors 234 may cooperatively locate one or more geometric features of the adapter cuff 200 in three-dimension space using the IGS navigation system 50 without requiring one of the single axis sensors 234 to be coaxial with the longitudinal axis LA1 of the shaft assembly 120.

Additionally, as shown in FIG. 5, the angular spacing A1, A2, A3 between adjacent single axis sensors 234 is not each 120 degrees (i.e., substantial equiangular), when viewed from the distal end as shown in FIG. 5. In the present example, angular spacing Al is approximately 180 degrees, angular spacing A2 is approximately 90 degrees, and angular spacing A3 is approximately 90 degrees. Alternatively, adjacent single axis sensors 234 may have any other suitable angular spacing A1, A2, A3, when viewed from the distal end as shown in FIG. 5, as would be apparent to one skilled in the art in view of the teachings herein.

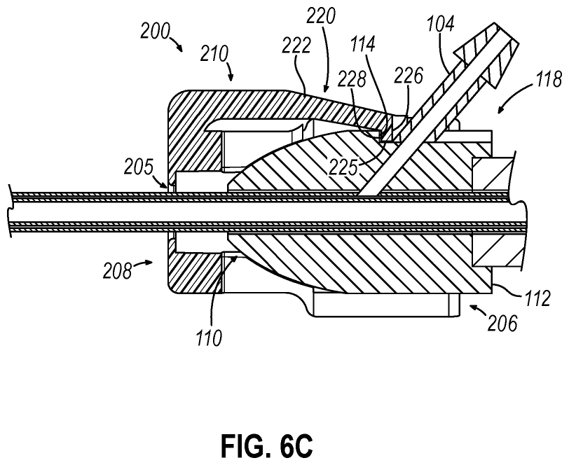

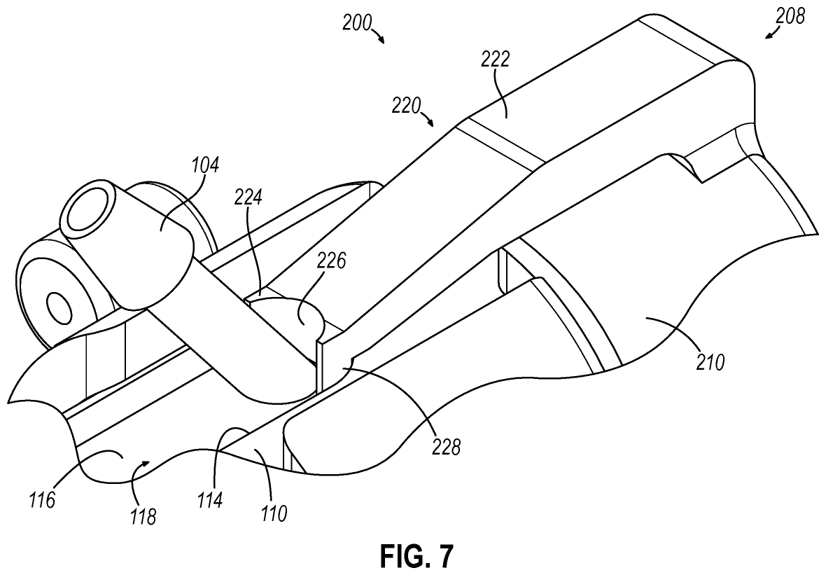

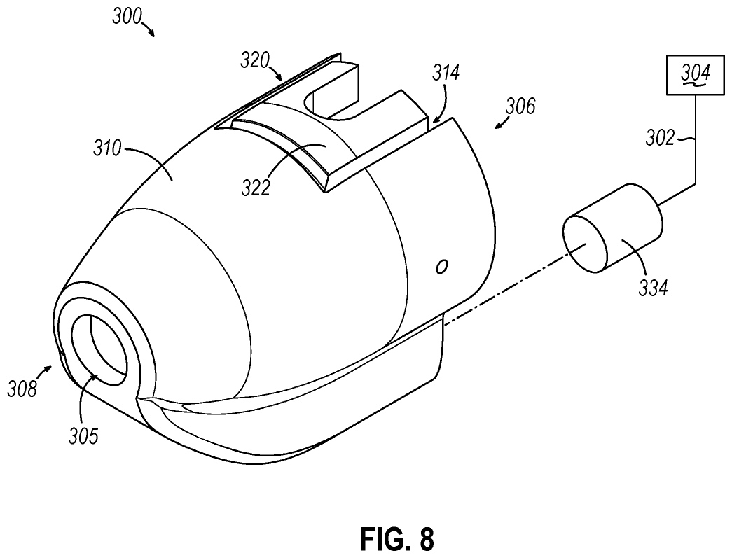

FIGS. 8-12 show an alternative the adapter cuff 300 that may be readily used in conjunction with the instrument 100 in replacement of the adapter cuff 200 described above. The adapter cuff 300 is substantially similar to the adapter cuff 200 described above, with differences elaborated below. Therefore, the adapter cuff 300 is generally configured to mate with the tissue shaving instrument 100 in such a way to consistently orient the adapter cuff 300 relative to the tissue shaving instrument 100; as well as prevent decoupling of the adapter cuff 300 from the tissue shaving instrument 100. Once suitably coupled, the adapter cuff 300 may add, or otherwise supplement, navigation capabilities with respect to the tissue shaving instrument 100. Thus, a position sensor assembly 330 of the adapter cuff 300 is configured for use in combination with the IGS navigation system 50.

The adapter cuff 300 of the presented example includes a cuff body 310, a coupling arm 320, the position sensor assembly 330, a cable 302, and a connector 304; which are substantially similar to the cuff body 210, the coupling arm 220, the position sensor assembly 230, the cable 202, and the connector 204, respectfully, with differences described herein.

Turning to FIGS. 8-10, the cuff body 310 extends between a proximal end 305 and a distal end 308. The cuff body 310 also defines a through hole 305 extending between the proximal end 305 and the distal end 308. Similar to the through hole 205 described above, the through hole 305 is dimensioned to receive the shaft assembly 120 in order to couple the adapter cuff 300 with the instrument 100 such that the distal end 308 of the cuff body 310 is adjacent to a proximal end of the shaft assembly 120, while proximal end 305 of the cuff body 310 suitably houses at least a portion of the distal hub 110 of the instrument 100.

The cuff body 310 also defines an arm housing window 314, which the hub coupling arm 320 is disposed within. Similar to the hub coupling arm 220 described above, the hub coupling arm 320 of the current example is configured to engage the distal hub 110 of the instrument 100 in order to consistently orient the adapter cuff 300 relative to the tissue shaving instrument 100, as well as inhibit the adapter cuff 300 from decoupling with the distal hub 110.

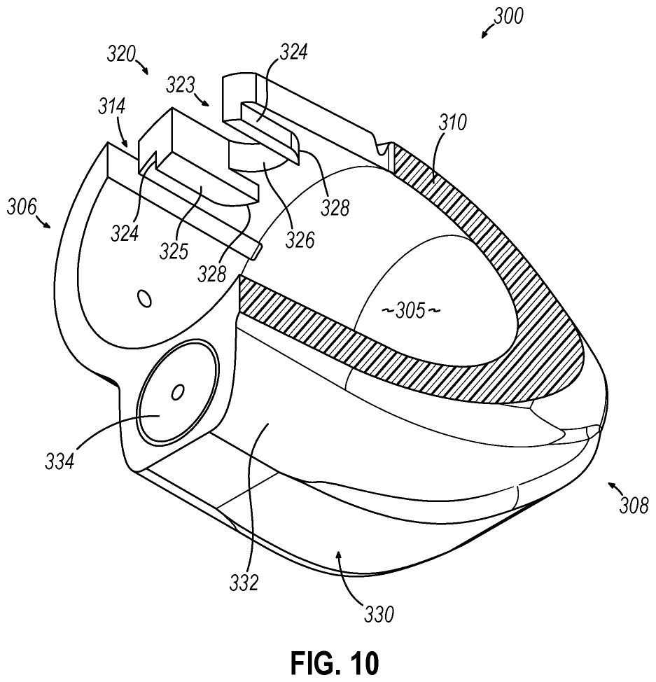

The hub coupling arm 320 includes a resilient arm 322 that extends proximally from a distal portion of the cuff body 310 defining the arm housing window 314. The resilient arm 322 of the current example defines an elongate slot 323 having a proximal open end. A port engagement surface 326 is located at a distal end of the elongate slot 323 such that the port engagement surface 326 partially defines the elongate slot 323. Two opposing hub engagement sections 224 extend downwardly from lateral sides of the resilient arm 322 defining the elongate slot 323. The hub engagement sections 224 are dimensioned to fit within a suitable portion of the pocket recess 118 when the adapter cuff 300 is operatively attached to the distal hub 110 (sec FIG. 12). In the current example, each hub engagement section 224 includes a boundary engagement surface 328 and an offset floor engagement surface 325.

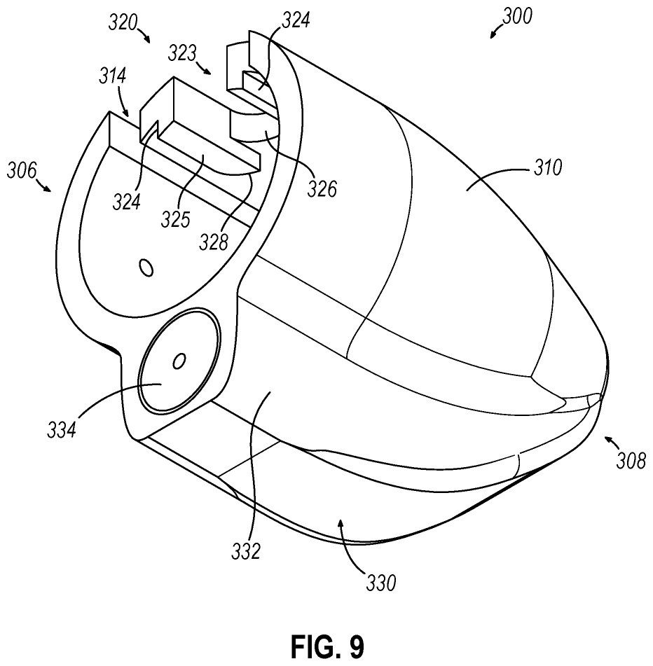

The resilient arm 322 is configured to deflect between a relaxed position (sec FIGS. 8-11A and 11C-12) and a flexed position (see FIG. 11B); and is resiliently biased toward the relaxed position. FIGS. 11A-11C show an example coupling of the adapter cuff 300 and the distal hub 110 of the instrument 100. First, as mentioned above, the proximal end 305 of the adapter cuff 300 may be slid over the end effector 122 and the shaft assembly 120 of the instrument 100 via the through hole 305 to the position shown in FIG. 11A. Next, the adapter cuff 300 may be further slid proximally as shown between FIGS. 11A-11B. When the adapter cuff 300 is inserted over the shaft assembly 120 onto the distal hub 110, the resilient arm 322 deflects into the flexed position in response to the distal hub engagement bodies 324 engaging the outer surface of the distal hub 110. As mentioned above, the hub engagement bodies 324 are dimensioned to at least partially rest within the recessed pocket 118 defined by the distal hub 110.

Once each hub engagement body 324 is longitudinally and laterally aligned with the recessed pocket 118, the resilient arm 322 may return toward the relaxed position, thereby driving hub engagement bodies 324 into the confines of the recessed pocket 118 as shown in FIGS. 11C and 12. Resilient arm 322 may drive each hub engagement body 324 into the confines of the recessed pocket 118 with sufficient force in order to provide tactile and/or audible feedback, thereby indicating to an operator that cuff adapter 300 is suitably engaged with the distal hub 110.

When the distal hub engagement body 324 is housed within the suitable portion of the pocket recess 118, as shown in FIG. 11C, at least a portion of the port engagement surface 226 is directly adjacent to the fluid port 104, while at least a portion of each recess the boundary engagement surface 328 is directly adjacent to the boundary surface 114 defining the recessed pocket 118. The offset floor engagement surface 325 may rest on the offset floor 116 of the distal hub 110 when the adapter cuff 200 is suitably coupled to the distal hub 110.

The port engagement surface 226 may contact the fluid port 104 in order to inhibit over insertion of the adapter cuff 200 proximally relative to the distal hub 110 when suitably coupled. Similarly, the distal ends of each hub engagement section may contact suitable portions of the boundary surface 114 defining the recessed pocket 118 in order to inhibit distal movement of the adapter cuff 300 relative to the distal hub 110 when suitably coupled. The port engagement surface 326 and/or each the boundary engagement surface 328 may also engage suitable portions of the fluid port 104 and/or the boundary surface 114, respectively, in order to inhibit the adapter cuff 300 from rotating about the longitudinal axis LA1 of the instrument 100. Therefore, once the adapter cuff 300 is suitably coupled to the distal hub 110, the cuff 300 may be constrained to the consistent and predetermined orientation relative to the distal hub 110 and inhibited from decoupling from the distal hub 110 via portions of the distal hub engagement body 324 housed within the recessed pocket 118 and the port engagement surface 226.

Ensuring that the adapter cuff 300 couples with hub 110 in only one predetermined angular orientation, while also inhibiting the adapter cuff 300 from moving relative to the hub 110 during an example of use, ensures sensors 335, 336, 338 consistently have a particular alignment with the end effector 122. Such a particular alignment may be desirable to enhance locating the end effector 122, and particularly the first transverse opening 132, in three-dimensional space via sensors 335, 336, 338 while using the IGS navigation system 50 in accordance with the description herein.

As shown in FIGS. 8 and 11A-11C, the navigation sensor assembly 330 includes a sensor housing 332 coupled to a concentric tri-axial sensor 334 having two annular sensors 336, 338 disposed around a central spherical sensor 335. The sensor housing 332 is fixedly attached to the cuff body 310 such that concentric the tri-axial sensor 334 is also fixed relative to the cuff body 310. The tri-axial sensor 334 may be secured to the sensor housing 332 using any suitable means as would be apparent to those skilled in the art in view of the teachings herein. For example, the tri-axial sensor 334 may be inserted into an opening defined by sensor housing 334 and fixed via adhesive, friction fitting, or both.

The cable 302 and the connector 304 are substantially similar to the cable 202 and the connector 204 described above. The cable 302 is coupled to each sensor 335, 336, 338. Therefore, the cable 302 and the connector 304 establish operative communication between tri-axis sensor 334 and the IGS navigation system 50 during an example of use in accordance with the description herein. Once the adapter cuff 300 is suitably attached to the instrument 100, the sensors 335, 336, 338 of the tri-axis sensor 334 cooperatively function to locate one or more geometric features of the adapter cuff 300 in three-dimensional space using the IGS navigation system 50. With the adapter cuff 300 being consistently positioned relative to the distal hub 110 and the instrument 100 via the hub coupling arm 320, the IGS navigation system 50 may utilize the location of the adapter cuff 300 to correlate the position of one or more features of tissue saving instruments 100 (e.g., the end effector 122, and more particularly the first transverse opening 132, etc.) to likewise locate one or more features of the tissue shaving instrument 100 in three-dimensional space.

Therefore, the tri-axis sensor 334 may be used with the IGS navigation system 50 to navigate the tissue shaving instrument 100 through one or more anatomical passageways of a patient and/or relative to one or more anatomical features of a patient. As described above, such navigation may be combined with navigation of other instruments (e.g., probes) to facilitate real-time overlays of instrument positioning on visualizations. In some uses, such real-time overlays may be used to facilitate true anatomical measurements of patient anatomy, thereby improving procedure workflows and procedure outcomes.

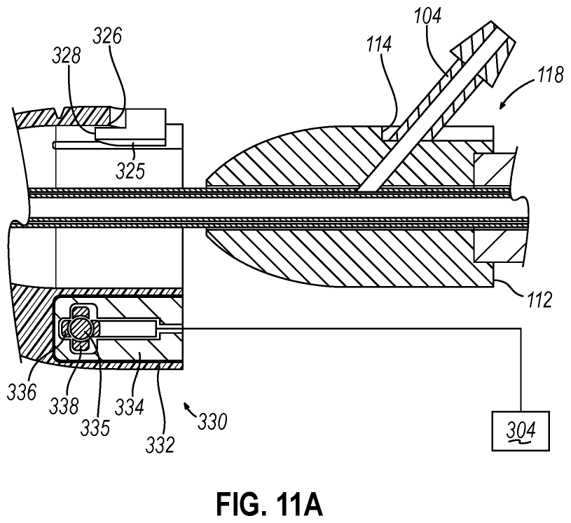

FIG. 13 shows another example of an adapter cuff 400 that is substantially similar to the adapter cuff 200, 300 described above, with differences elaborated below. The adapter cuff 400 includes a cuff body 402, a hub coupling arm 404, and a sensor assembly 406 that are substantially similar to the cuff body 210, 310, the hub coupling arm 220, 320, and the sensor assembly 230, 330 described above, with differences elaborated herein. In particular, the adapter cuff 400 of the present example includes a sensor assembly 406 having a standard tri-axis sensor assembly rather than the concentric tri-axis sensor assembly 330 or multiple single axis sensor assembly 230 described above.

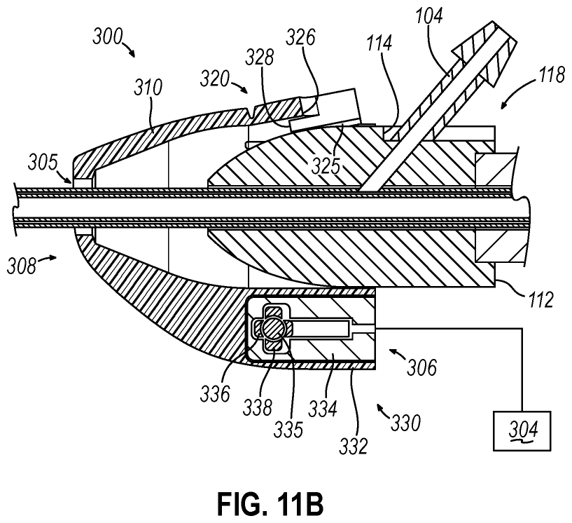

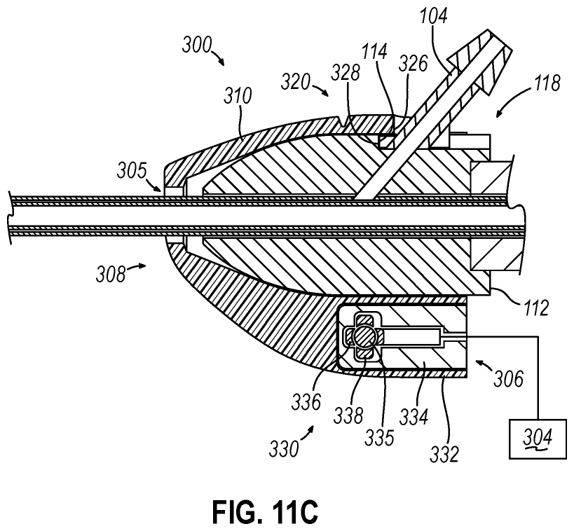

FIGS. 14-15 show another example of an adapter cuff 500 that is substantially similar to the adapter cuff 200, 300, 400 described above, with differences elaborated below. The adapter cuff 500 includes a cuff body 502, a hub coupling arm 504, and a sensor assembly 506 that are substantially similar to the cuff body 210, 310, the hub coupling arm 220, 320, and the sensor assembly 230, 330 described above, with differences elaborated herein. In particular, the adapter cuff 500 of the present example includes a sensor assembly 506 having three 3D printed circuit sensors 508, 510 positioned within portions of cuff body 502 rather than the concentric tri-axis sensor assembly 330 or the multiple single axis sensor assembly 230 described above. Two 3D printed circuit sensors 508 are disposed adjacent to the proximal end of the cuff body 502 as shown in FIG. 14, while a third annular 3D printed circuit sensor 510 is disposed adjacent to the distal end of the cuff body 502 for coaxial receipt of the shaft assembly 120. The 3D printed circuit sensors 508, 510 may be used in conjunction with IGS navigation system in substantially similar manner to the sensors 234, 334 described above.

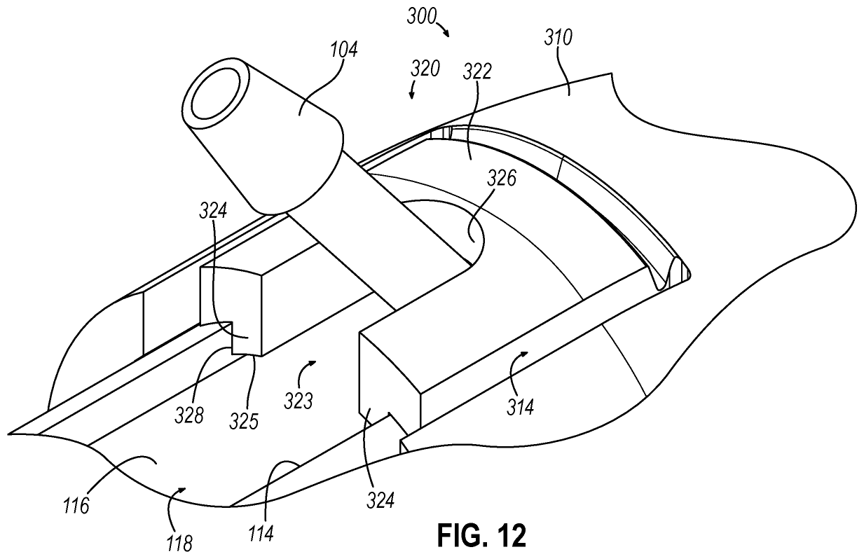

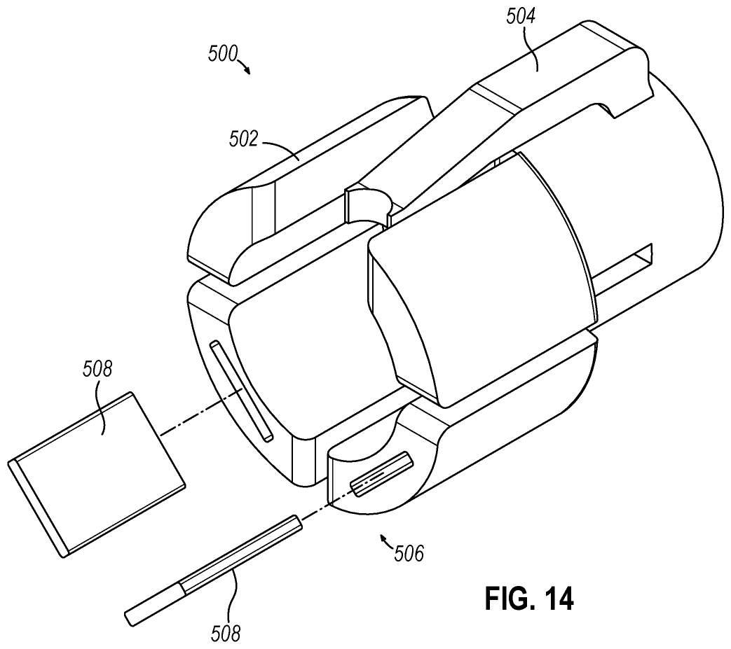

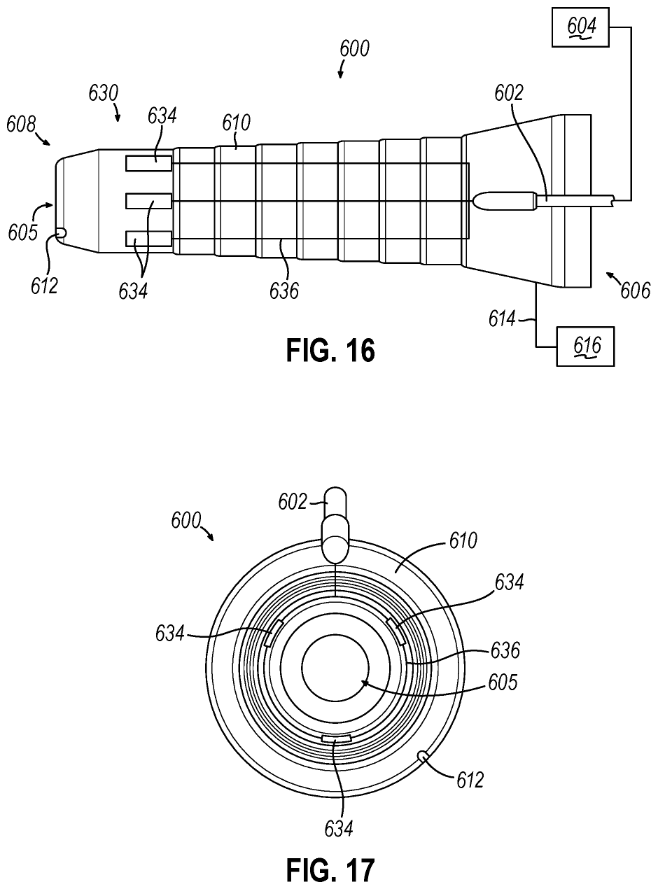

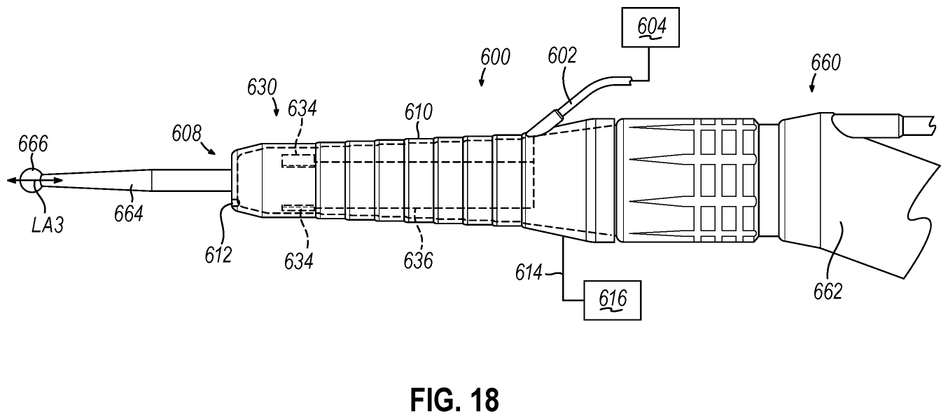

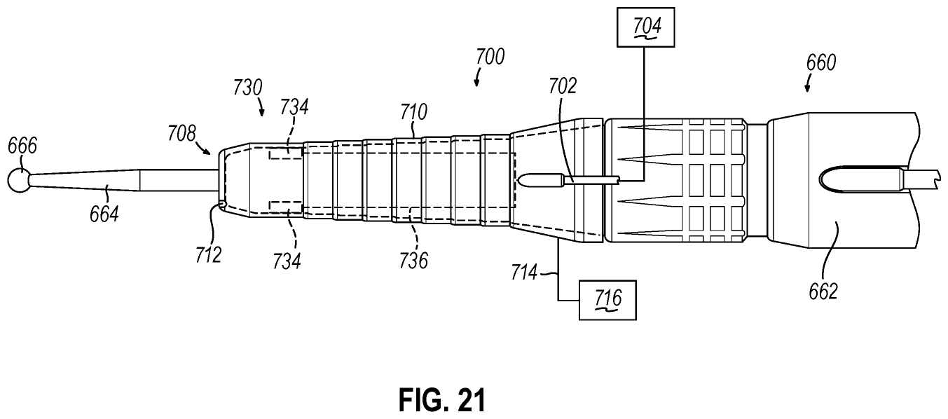

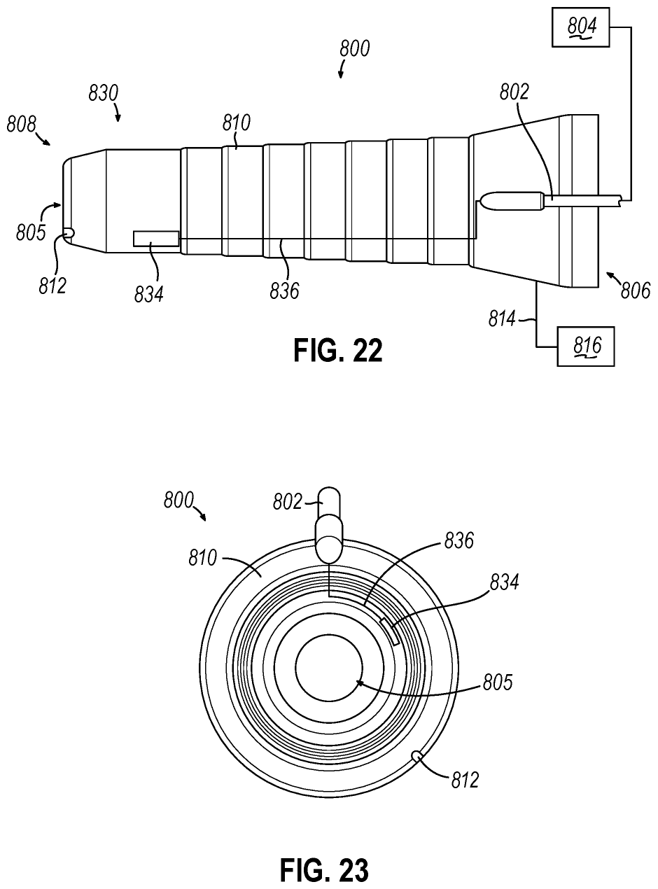

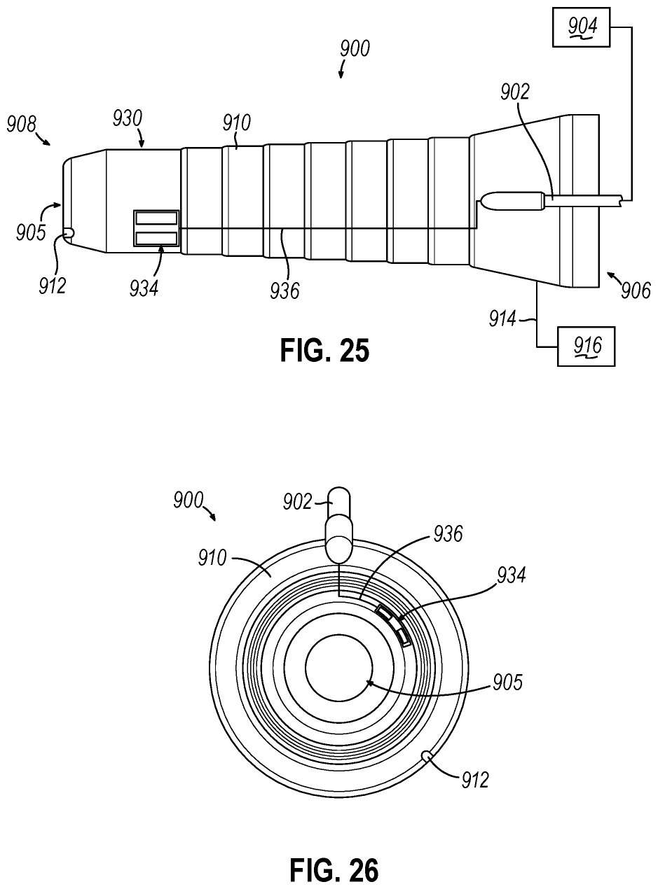

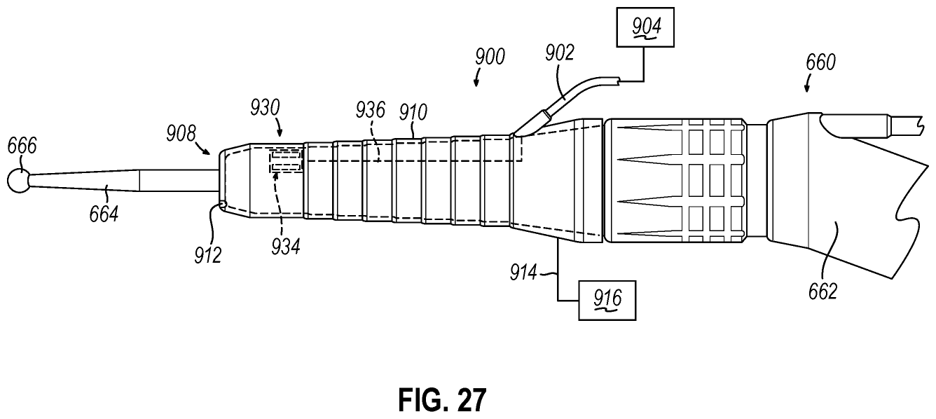

As mentioned above, a navigation adapter such as those described herein may be configured to accommodate different kinds of instruments and/or instruments otherwise having different outer diameters. FIGS. 16-27 show various adapters 600, 700, 800, 900 having navigation capabilities similar to those of the adapters 200, 300, 400, 500 described above, but for use with a drill instrument 660 (see FIGS. 18, 21, 24, and 27). As will also be described in greater detail below, each the adapter 600, 700, 800, 900 is configured to provide fluid irrigation and/or suction at or near the distal end of the drill instrument 660.

The drill instrument 660 of the current example includes a proximal handle 662, an elongate shaft 664 extending distally from the proximal handle 662, and a drill bit 666 located at the distal end of the elongate shaft 664. The elongate shaft 664 extends away from the proximal handle 662 along a substantially linear profile along longitudinal axis (LA3) of the elongate shaft 664. The drill bit 666 is also located on a distal end of the elongate shaft 664 at a location that is coaxial with longitudinal axis (LA3) of the elongate shaft 664. The drill instrument 660 may have any suitable components required for a user to activate the drill bit 666 in order to drill suitable anatomical structures as would be apparent to one skilled in the art in view of the teachings herein.

The adapter 600, as shown in FIGS. 16-18, includes an adapter body 610, a position sensor assembly 630, a cable 602, a connector 604, and a fluid communication line 614 configured to suitably couple to a fluid source 616. The adapter body 610 extends from a proximal end 606 to a distal end 608. The adapter body 610 defines a through hole 605 that extends along the length of the body 610 and through both the proximal end 606 and the distal end 608. The through hole 605 is dimensioned to receive the elongate shaft 664 in order to couple the adapter 600 with the instrument 660 such that the proximal end 606 of the adapter body 610 abuts against (or is directly adjacent to) the proximal handle 662. Therefore, an operator may insert the distal end of the elongate shaft 664 into the through hole 605 via the proximal end 606 of the adapter body 610 and actuate the adapter body 610 proximally relative to the elongate shaft 664. The proximal end 606 may include any suitable engagement features in order to ensure the adapter 600 remains operatively engaged with the drill instrument 660 during an example of use. For example, the proximal end 606 may include a friction fit feature configured to keep the adapter 600 substantially fixed relative to the instrument 660 during an example of use in accordance with the description herein. Therefore, while the adapter 600 is suitable engaged with the instrument 660, an operator may control the placement of the instrument 660 by grasping and controlling the adapter body 610.

The distal end 608 of the adapter body 610 includes an irrigation nozzle 612. The adapter body 610 defines an irrigation lumen that extends proximally from the irrigation nozzle 612 and is in fluid communication with the irrigation line 614. Therefore, the irrigation source 616 may communicate fluid to the irrigation nozzle 612 via the irrigation line 614 and the irrigation lumen defined by the adapter body 610. The irrigation nozzle 612 is configured to direct irrigation fluid toward the drill bit 666 when the adapter 600 is operatively coupled with the drill instrument 660. In some instances, the irrigation nozzle 612 may be used to apply suction to a location adjacent to the drill bit 666 during use.

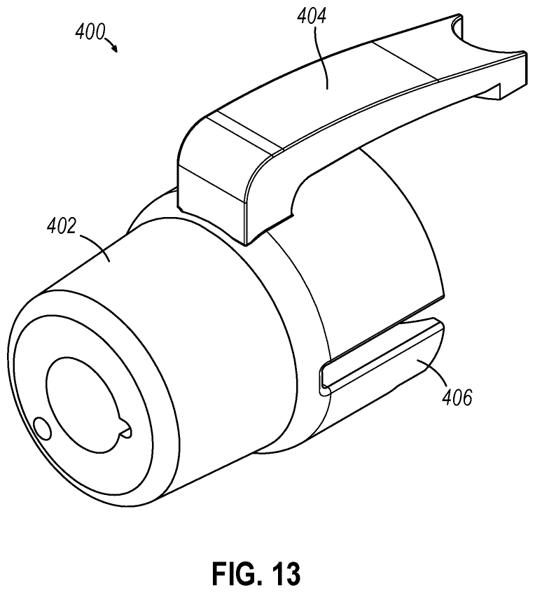

The navigation sensor assembly 630 includes three single axis sensors 634 embedded into the distal end 608 of the adapter body 610. Each single axis sensor 634 is secured to the adapter body 610 via any suitable means as would be apparent to those skilled in the art in view of the teachings herein. As best shown in FIG. 17, the single axis sensors 634 are disposed circumferentially around the adapter body 610. The single axis sensors 634 are in communication with the cable 602 via electrical traces 636 extending through the adapter body 610. However, the single axis sensors 634 may be in communication with the cable 602 using any suitable means as would be apparent to one skilled in the art in view of the teachings herein.

The single axis sensors 634 are in communication with the connector 604 via the cable 602. The connector 604 is configured to facilitate communication of the single axis sensors 634 with the console 58 of the IGS navigation system 50. Therefore, during an example of use, the single axis sensors 634 are in operative communication with the IGS navigation system 50 via the cable 602 and the connector 604. The connector 604 may establish communication with the console 58 via wired and/or wireless communication. Once the adapter 600 is suitably attached to the instrument 660, the three single axis sensors 634 cooperatively function to locate one or more geometric features of the adapter 600 in three-dimensional space using the IGS navigation system 50. The IGS navigation system 50 may utilize the location of the adapter 600 to correlate the position of one or more features of the instrument 660 (e.g., the drill bit 666, etc.) to likewise locate one or more features of the instrument 660 in three-dimensional space.

Therefore, the navigation sensors 634 may be used with the IGS navigation system 50 to navigate instrument 660 through one or more anatomical passageways of a patient and/or relative to one or more anatomical features of a patient. As described above, such navigation may be combined with navigation of other instruments (e.g., probes) to facilitate real-time overlays of instrument positioning on visualizations. In some uses, such real-time overlays may be used to facilitate true anatomical measurements of patient anatomy, thereby improving procedure workflows and procedure outcomes.

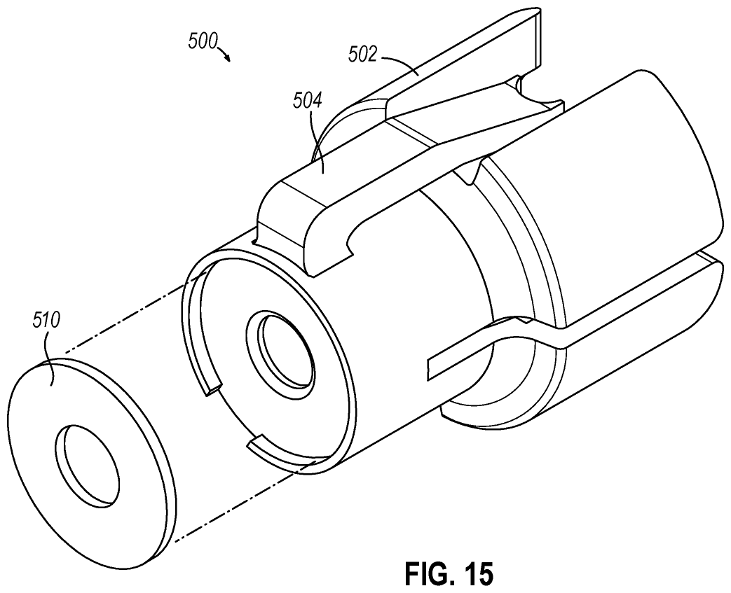

FIGS. 19-21 show another the adapter 700 having navigation capabilities and configured to be used with the drill instrument 660. The adapter 700 is substantially similar to the adapter 600 described above, except for differences elaborated below. The adapter 700 of this example includes a cable 702, a connector 704, an adapter body 710 defining a through hole 705 and extending between a proximal end 706 and a distal end 708, an irrigation nozzle 712, an irrigation line 714, and an irrigation source 716; which may be substantially similar to the cable 602, the connector 604, the adapter body 610, the through hole 605, the proximal end 606, the distal end 608, the irrigation nozzle 612, the irrigation line 614, and irrigation source 616 described above.

The adapter 700 also includes a sensor assembly 730. However, the sensor assembly 730 of the current example includes two single axis sensors 734 embedded in the adapter body 710. Each single axis sensor 734 is in communication with the cable 702 via electrical traces 736. However, the single axis sensors 734 may be in communication with the cable 702 using any suitable means as would be apparent to one skilled in the art in view of the teachings herein.

Once the adapter 700 is suitably attached to the instrument 660, the two single axis sensors 734 cooperatively function to locate one or more geometric features of the adapter 700 in two-dimensional space using the IGS navigation system 50. The IGS navigation system 50 may utilize the location of the adapter 700 to correlate the position of one or more features of the instrument 660 (e.g., the drill bit 666, etc.) to likewise locate one or more features of the instrument 660 in two-dimensional space.

Therefore, the navigation sensors 734 may be used with the IGS navigation system 50 to navigate instrument 660 through one or more anatomical passageways of a patient and/or relative to one or more anatomical features of a patient. As described above, such navigation may be combined with navigation of other instruments (e.g., probes) to facilitate real-time overlays of instrument positioning on visualizations. In some uses, such real-time overlays may be used to facilitate true anatomical measurements of patient anatomy, thereby improving procedure workflows and procedure outcomes.

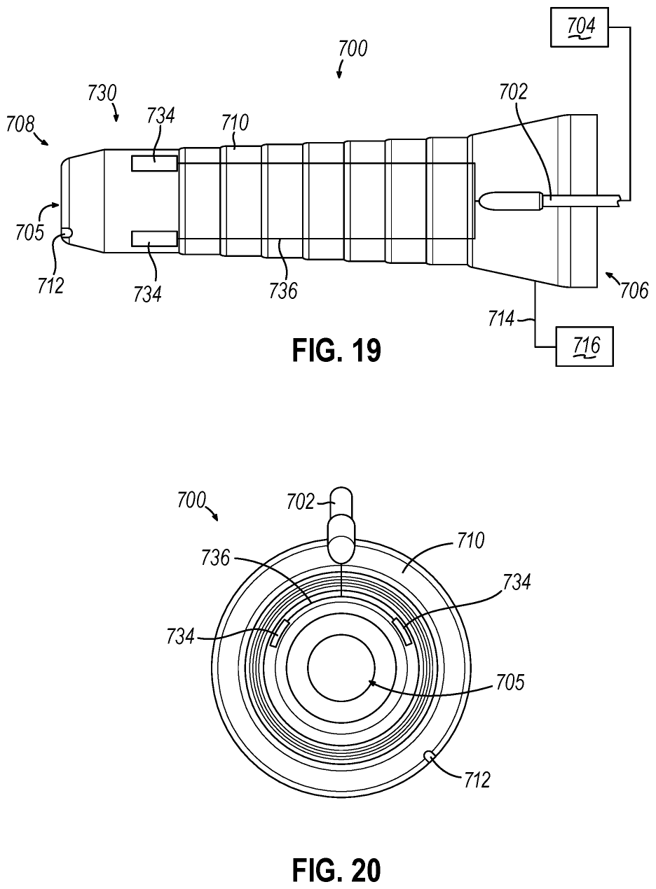

FIGS. 22-24 show another the adapter 800 having navigation capabilities and configured to be used with the drill instrument 660. The adapter 800 is substantially similar to the adapter 600 described above, except for differences elaborated below. The adapter 800 of this example includes a cable 802, a connector 804, an adapter body 810 defining a through hole 805 and extending between a proximal end 806 and a distal end 808, an irrigation nozzle 812, an irrigation line 814, and an irrigation source 816; which may be substantially similar to the cable 602, the connector 604, the adapter body 610, the through hole 605, the proximal end 606, the distal end 608, the irrigation nozzle 612, the irrigation line 614, and irrigation source 616 described above.

The adapter 800 also includes a sensor assembly 830. However, the sensor assembly 830 of the current example includes a non-concentric triple axis sensor 834 embedded in the adapter body 810. The non-concentric triple axis sensor 834 is in communication with the cable 802 via the electrical traces 836. However, the non-concentric triple axis sensor 834 may be in communication with the cable 802 using any suitable means as would be apparent to one skilled in the art in view of the teachings herein.

Once the adapter 800 is suitably attached to the instrument 660, the non-concentric triple axis sensor 834 functions to locate one or more geometric features of the adapter 800 in three-dimensional space using the IGS navigation system 50. The IGS navigation system 50 may utilize the location of the adapter 800 to correlate the position of one or more features of the instrument 660 (e.g., the drill bit 666, etc.) to likewise locate one or more features of the instrument 660 in two-dimensional space.

Therefore, the non-concentric triple axis sensor 834 may be used with the IGS navigation system 50 to navigate the instrument 660 through one or more anatomical passageways of a patient and/or relative to one or more anatomical features of a patient. As described above, such navigation may be combined with navigation of other instruments (e.g., probes) to facilitate real-time overlays of instrument positioning on visualizations. In some uses, such real-time overlays may be used to facilitate true anatomical measurements of patient anatomy, thereby improving procedure workflows and procedure outcomes.

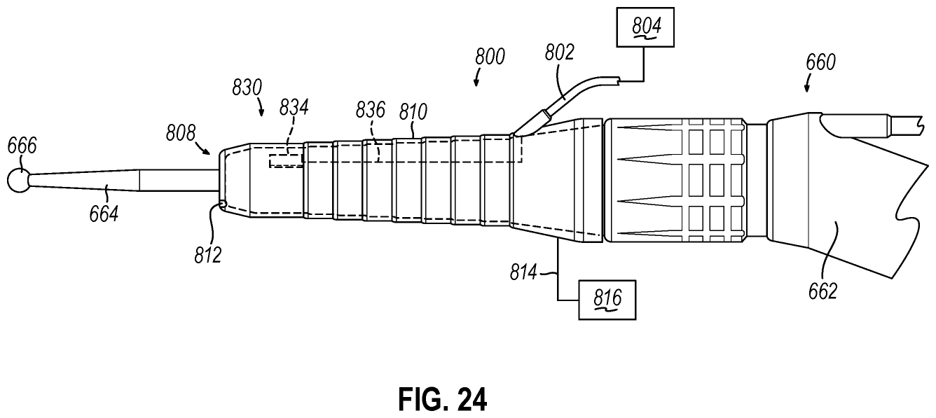

FIGS. 25-27 show another the adapter 900 having navigation capabilities and configured to be used with the drill instrument 660. The adapter 900 is substantially similar to the adapter 600 described above, except for differences elaborated below. The adapter 900 of this example includes a cable 902, a connector 904, an adapter body 910 defining a through hole 905 and extending between a proximal end 896 and a distal end 908, an irrigation nozzle 912, an irrigation line 914, and an irrigation source 916; which may be substantially similar to the cable 602, the connector 604, the adapter body 610, the through hole 605, the proximal end 606, the distal end 608, the irrigation nozzle 612, the irrigation line 614, and irrigation source 616 described above.

The adapter 900 also includes a sensor assembly 930. However, the sensor assembly 930 of the current example includes a single dual axis sensor 934 embedded in adapter body 910. The dual axis sensor 934 is in communication with the cable 902 via the electrical traces 936. However, the dual axis sensor 934 may be in communication with the cable 902 using any suitable means as would be apparent to one skilled in the art in view of the teachings herein.

Once the adapter 900 is suitably attached to the instrument 660, dual axis sensor 734 functions to locate one or more geometric features of the adapter 700 in two-dimensional space using the IGS navigation system 50. The IGS navigation system 50 may utilize the location of the adapter 900 to correlate the position of one or more features of the instrument 660 (e.g., the drill bit 666, etc.) to likewise locate one or more features of the instrument 660 in two-dimensional space.

Therefore, the navigation dual axis sensor 934 may be used with the IGS navigation system 50 to navigate the instrument 660 through one or more anatomical passageways of a patient and/or relative to one or more anatomical features of a patient. As described above, such navigation may be combined with navigation of other instruments (e.g., probes) to facilitate real-time overlays of instrument positioning on visualizations. In some uses, such real-time overlays may be used to facilitate true anatomical measurements of patient anatomy, thereby improving procedure workflows and procedure outcomes.

### IV. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

Example 1: An apparatus for use with an instrument, the apparatus comprising: (a) an adapter body extending between a proximal end and a distal end; (b) an instrument coupling arm attached to the adapter body, the instrument coupling arm configured to non-removably engage a portion of the instrument; and (c) one or more navigation sensors fixed to the adapter body, the one or more navigation sensors being configured generate signals indicating a position of at least a portion of the instrument in three-dimensional space.

Example 2: The apparatus of Example 1, the one or more navigation sensors including a plurality of navigation sensors.

Example 3: The apparatus of Example 2, the plurality of navigation sensors comprising a first single axis sensor, a second single axis sensor, and a third single axis sensor.

Example 4: The apparatus of Example 3, neither the first single axis sensor, the second single axis sensor, nor the third single axis sensor being coaxial with a longitudinal axis of a shaft of the instrument while the instrument coupling arm is non-removably engaged with the portion of the instrument.

Example 5: The apparatus of Example 4, neither of the first single axis sensor, the second single axis sensor, nor the third single axis sensor being angularly spaced apart from each other by 120 degrees about the longitudinal axis of the shaft of the instrument while the instrument coupling arm is non-removably engaged with the portion of the instrument.

Example 6: The apparatus of Example 2, the plurality of navigation sensors comprising a first annular sensor, a second annular sensor, and a spherical sensor.

Example 7: The apparatus of Example 6, the first annular sensor and the second annular sensor being disposed around the spherical sensor.

Example 8: The apparatus of any of Examples 1 through 7, the instrument coupling arm comprising a resilient member.

Example 9: The apparatus of Example 8, the instrument coupling arm comprising a complementary surface configured to rest within a recess defined by the portion of the instrument.

Example 10: The apparatus of Example 9, the complementary surface having an arched profile.

Example 11: The apparatus of Example 8, the resilient member defining an elongate slot.

Example 12: The apparatus of any of Examples 8 through 11, the resilient member extending proximally from the distal end of the adapter body.

Example 13: The apparatus of any of Examples 1 through 12, the instrument coupling arm configured to non-removably engage a portion of the instrument, in only a predetermined angular orientation with respect to an axis of the instrument.

Example 14: The apparatus of any of Examples 1 through 13, the adapter body defining a through hole extending between the proximal end and the distal end.

Example 15: The apparatus of any of Examples 1 through 14, the one or more navigation sensors comprising a 3D printed circuit sensor.

Example 16: An assembly for use in an ENT procedure, the assembly comprising: (a) a rotary instrument, the rotary instrument including: (i) a body, (ii) a hub associated with the body, and (iii) a shaft extending from a portion of the body or the hub, the shaft including an end effector proximate a distal end of the shaft, the end effector including a rotating feature, the rotating feature being configured to affect tissue; and (b) an adapter, the adapter including: (i) an adapter body configured to non-removably receive a portion of the hub of the tissue shaving instrument at only a predetermined angular orientation about a longitudinal axis of the shaft, and (ii) one or more navigation sensors secured to a portion of the adapter body, each navigation sensor being configured to generate signals indicating a position of the end effector in three-dimensional space.

Example 17: The assembly of Example 16, the hub of the rotary instrument defining a recessed pocket and including a fluid port, extending outward from the recessed pocket, the adapter body comprising a hub engagement body being complementary to the recessed pocket adjacent to the fluid port such that the recessed pocket is dimensioned to receive the hub engagement body.

Example 18: The assembly of Example 17, the hub engagement body being dimensioned to engage the fluid port once the adapter body non-removably receives the portion of the hub.

Example 19: An apparatus for use with an instrument, the apparatus comprising: (a) an adapter body extending between a proximal end and a distal end, the adapter body defining an irrigation lumen extending from the distal end toward the proximal end; (b) an irrigation nozzle located at the distal end of the adapter body and in fluid communication with the irrigation lumen; and (c) one or more navigation sensors fixed to the adapter body, the one or more navigation sensors being configured generate signals indicating a position of at least a portion of the instrument.

Example 20: The apparatus of Example 19, the one or more navigation sensors comprising three single axis sensors located near the distal end of the adapter body.

Example 21: The apparatus of Example 19, the one or more navigation sensor comprising two single axis sensors located near the distal end of the adapter body.

Example 22: The apparatus of Example 19, the one or more navigation sensors comprising a dual axis sensor.

Example 23: The apparatus of Example 19, the one or more navigation sensor comprising a non-concentric tri-axis sensor.

### V. Miscellaneous

It should be understood that any of the teachings, expressions, embodiments, examples, etc. described herein may be combined with any of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present inventions, further adaptations of the methods and systems described herein may be accomplished by appropriate modifications by one skilled in the art without departing from the scope of the present inventions. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present inventions should be considered in terms of the following claims and is understood not to be limited to the details of structure and operation shown and described in the specification and drawings.

What is claimed is:

1. An adapter for use with an instrument, the adapter comprising:
an adapter body extending between a proximal end and a distal end;
an instrument coupling arm attached to the adapter body, the instrument coupling arm configured to non-removably engage a portion of the instrument; and
a navigation sensor fixed to the adapter body, the navigation sensor configured generate signals indicating a position of at least a portion of the instrument in three-dimensional space.

2. The adapter of claim 1, wherein the of navigation sensor comprises a first single axis sensor, a second single axis sensor, and a third single axis sensor that are configured to be non-coaxial with a longitudinal axis of a shaft of the instrument when the instrument coupling arm is non-removably engaged with the portion of the instrument.

3. The adapter of claim 2, wherein, when the instrument coupling arm is non-removably engaged with the portion of the instrument, the first single axis sensor, the second single axis sensor, and the third single axis sensor are configured to be angularly offset relative to each other by 120 degrees about the longitudinal axis of the shaft of the instrument.

4. The adapter of claim 1, wherein the navigation sensor comprises a first annular sensor, a second annular sensor, and a spherical sensor, the first annular sensor and the second annular sensor being disposed around the spherical sensor.

5. The adapter of claim 1, wherein the instrument coupling arm comprises a resilient member.

6. The adapter of claim 5, wherein the instrument coupling arm comprises a complementary surface configured to rest within a recess defined by the portion of the instrument.

7. The adapter of claim 6, wherein the complementary surface comprises an arched profile.

8. The adapter of claim 5, wherein the resilient member defines an elongate slot.

9. The adapter of claim 5, wherein the resilient member extends proximally from the distal end of the adapter body.

10. The adapter of claim 1, wherein the instrument coupling arm is configured to non-removably engage a portion of the instrument, in a predetermined angular orientation with respect to an axis of the instrument.

11. The adapter of claim 1, wherein the adapter body defines a through hole extending between the proximal end and the distal end.

12. The adapter of claim 1, wherein the navigation sensor comprises a 3D printed circuit sensor.

13. An assembly for use in an ENT procedure, the assembly comprising:
a rotary instrument comprising a body, a hub associated with the body, and a shaft extending from a portion of the body or the hub, wherein the shaft comprises an end effector proximate a distal end of the shaft, the end effector comprising a rotating feature configured to affect tissue; and
an adapter comprising an adapter body configured to non-removably receive a portion of the hub of the rotary instrument at a predetermined angular orientation about a longitudinal axis of the shaft, and a navigation sensor secured to a portion of the adapter body, the navigation sensor being configured to generate signals indicating a position of the end effector in three-dimensional space.

14. The assembly of claim 13, wherein the hub of the rotary instrument defines a recessed pocket and comprises a fluid port that extends outward from the recessed pocket, and wherein the adapter body comprises a hub engagement body that is complementary to the recessed pocket and adjacent to the fluid port such that the recessed pocket is dimensioned to receive the hub engagement body.

15. The assembly of claim 14, wherein the hub engagement body is dimensioned to engage the fluid port once the adapter body non-removably receives the portion of the hub.

16. An adapter cuff for use with an instrument, the adapter cuff comprising:
an adapter body extending between a proximal end and a distal end, the adapter body defining an irrigation lumen extending from the distal end toward the proximal end;
an irrigation nozzle located at the distal end of the adapter body and in fluid communication with the irrigation lumen; and
a navigation sensor fixed to the adapter body, the navigation sensor being configured generate signals indicating a position of at least a portion of the instrument.

17. The adapter cuff of claim 16, wherein the navigation sensor comprises three single axis sensors located near the distal end of the adapter body.

18. The adapter cuff of claim 16, wherein the navigation sensor comprises two single axis sensors located near the distal end of the adapter body.

19. The adapter cuff of claim 16, wherein the navigation sensor comprises a dual axis sensor.

20. The adapter cuff of claim 16, wherein the one or more navigation sensor comprises a non-concentric tri-axis sensor.
